(19) United States
(12) Patent Application Publication
(10) Pub. No.: US 2011/0224579 A1
(43) Pub. Date: Sep. 15, 2011

(54) DEVICE FOR COLLECTING A BIOLOGICAL FLUID SAMPLE

(75) Inventors: Joost H. Maas, Eindhoven (NL); Petrus L.A. Van Der Made, Boxtel (NL); Albert H.J. Immink, Eindhoven (NL)

(73) Assignee: KONINKLIJKE PHILIPS ELECTRONICS N.V., EINDHOVEN (NL)

(21) Appl. No.: 13/128,975

(22) PCT Filed: Nov. 10, 2009
(86) PCT No.: PCT/IB2009/054977

§ 371 Date: May 12, 2011

(30) Foreign Application Priority Data
Nov. 17, 2008 (EP) 08305806.5

(51) Int. Cl. A61B 5/00

(52) U.S. Cl. 600/573

(57) ABSTRACT

Sample collection device (10; 20; 30) for collecting a fluidic biological sample, the device comprising a collecting member (11; 21; 31) for collecting the fluidic sample; an indicator element (12, 22, 32) for providing an indication that a desired amount of the fluidic sample has been collected; an elongated capillary element (13; 23; 33) for providing fluidic communication between the collecting member (11; 21; 31) and the indicator element (12; 22; 32) so that at least a portion of the fluidic sample may be transferred from the collecting member (11; 21; 31) to the indicator element (12; 22; 32). The invention relates also to a sample test assembly.

### FIELD OF THE INVENTION

The present invention relates to a device for collecting a biological fluid sample. In particular the invention relates to a sample collection device provided with a sample sufficiency indicator. The invention further relates to a method of collecting a biological fluid sample, as well as an apparatus for and a method of analysing a biological fluidic sample so obtained.

### BACKGROUND OF THE INVENTION

Sample collection devices are used to collect biological fluids for analytical purposes. Such fluids may include saliva, urine, blood or other bodily fluids. Analysis of collected fluid samples may include testing for the presence or absence of specific substances such as certain drugs, proteins, genetic substances or DNA markers. A typical sample collector comprises means for readily absorbing the fluidic biological sample to be analysed and for readily releasing the fluid sample by for example mechanical pressure. The fluid sample can then be transferred to a counterpart of an analysing device for analysing the content of the fluidic sample.

In many applications it is important for the patient or the carer collecting the sample to be able to determine when a saturation level of a sample fluid has been reached or at least a minimum amount of sample sufficient for analysis purposes has been collected.

In an attempt to provide an indication of when a sufficient amount of a sample has been collected, some known sample collectors include indicator means for measuring sample adequacy. Such indicator means are arranged to be activated by a sample of liquid and, once activated, to indicate to the user of the sample collector when a sufficient quantity of sample has been collected. For example, the indication means may contain a visible chemical component which changes colour when the amount of sample collected is beyond a determinate threshold.

US2006/0018800 discloses a specimen sample collection device including such indication means. The device described in this document comprises a handle and a rounded absorbent pad tongue made of a spongeous material. One end of the tongue is exposed to collect the sample from a patient and the second opposing end of the tongue extends within the handle. The material from which the tongue is made is selected for its dispersion properties so that it can disperse a high volume of liquid sample over its entire surface. A portion of the second end portion of the tongue is coated with a visible chemical component which changes colour when a sufficient amount of sample has been collected and which may be viewed through a light pipe window provided on the wall of the handle.

### SUMMARY OF THE INVENTION

The inventors have realised that it would be advantageous to provide a device for collecting samples which requires a reduced amount of collected sample to indicate sample adequacy, and which requires less time to collect a sufficient sample amount, that it would also be desirable to provide a sample collection device which is less cumbersome than prior art devices, and that it would further be desirable to provide a method of collecting a sample which enables more rapid sample analysis.

Accordingly, a first aspect of the invention provides a sample collection device for collecting a fluidic biological sample, the device comprising: a collecting member for collecting the fluidic sample; an indicator element for providing an indication that a desired amount of the fluidic sample has been collected; and an elongated capillary element for providing fluidic communication between the collecting member and the indicator element such that at least a portion of the fluidic sample may be transferred from the collecting member to the indicator element. The fluidic sample is therefore at least partly transferred by capillary effect.

Optionally, the mean cross section of the elongated capillary is significantly less than the mean cross-section of the collecting member. The mean cross section of the collecting member may for example be more than ten times greater than the mean cross-section of the capillary element.

The capillary element is elongated such that it may extend along a single line.

The indicator element may provide an indication upon contact with the sample fluid, or upon reception of an activating amount of the sample fluid.

In an embodiment the capillary element comprises a tube having an inner bore arranged to provide a capillary effect. A porous material may be provided in at least part of the inner bore.

Alternatively the capillary element does not provide a capillary effect but includes a porous material providing a capillary effect between the collecting member and the indicator element.

In another alternative embodiment, the capillary element comprises a connecting element arranged between the collecting member and the indicator element, wherein the outer surface of the connecting element is provided with at least one capillary channel in fluidic communication between the collecting member and the indicator element. Additionally, the capillary element may comprise means for laterally closing at least one capillary channel, e.g. an outer shell surrounding the periphery of the connecting element. The connecting element may be a rigid elongated rod.

The sample collection device may further comprise a housing lodging at least the indicator element, the housing being provided with a window for viewing the indicator element.

In one or more embodiments the volume of the indicator element is significantly less than the volume of the collecting member.

The collecting member may be operable to release the fluidic sample by application of mechanical pressure. For example, by squeezing of the collecting member

In some embodiments of the invention, the device is operable to cooperate with a receiving member of a sample test device to transfer the fluidic sample into the sample test device. The device may further comprise retaining members for retaining the device in place relative to the sample test device.

The length of the capillary element may be chosen such that, in use, when a user collects the fluidic sample via the collecting member he can see the indicator element.

The sample collection device may be a saliva collection device for collecting saliva from an oral cavity.

According to a second aspect of the invention there is provided a sample test assembly comprising a receiving counterpart and the sample collection device according to any of the above embodiments, the receiving counterpart comprising a receiving portion arranged to receive the sample collection device and a sample collecting chamber having an inlet arranged to co-operate with the sample collection device to release the fluidic sample from the collecting member when the device is received in the receiving portion.

### BRIEF DESCRIPTION OF THE DRAWINGS

Embodiments of the invention will now be described, by way of example only, and with reference to the following drawings in which:—

FIG. 1A is a side view of the internal components of a sample collection device according to a first embodiment of the invention;

FIG. 1B is a cross-sectional view of the capillary channel of FIG. 1A along line IB;

FIG. 1C is an external perspective view of the sample collection device according to the first embodiment of the invention;

FIG. 2A is a schematic view of a sample collection device according to a second embodiment of the invention;

FIG. 2B is a partial perspective view of the connecting rod of the second embodiment of the invention;

FIG. 2C is a cross-sectional view of the connecting rod of the second embodiment along line IIB of FIG. 2A;

FIG. 3A is a perspective sectional view of a sample collection device according to a third embodiment of the invention; and

FIG. 3B is a cross-sectional view of the capillary channel of FIG. 3A along line IIIB.

### DETAILED DESCRIPTION

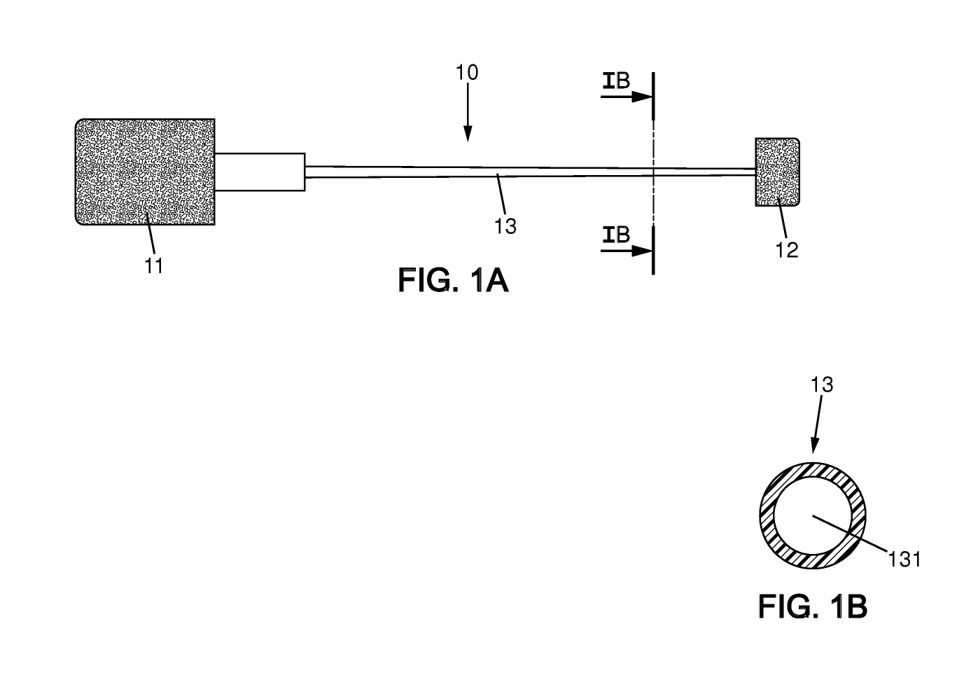

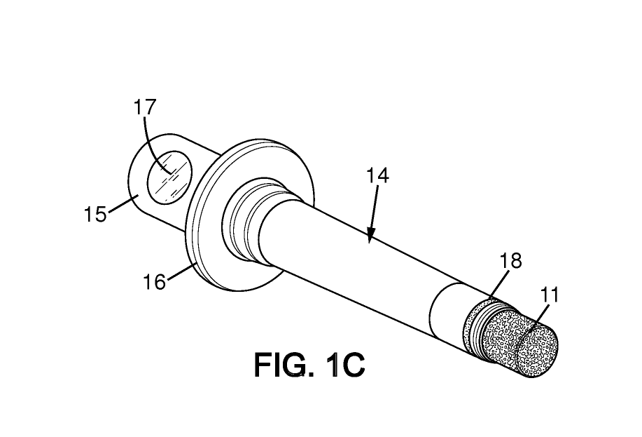

A sample collection device according to a first embodiment of the invention will be described with reference to FIGS. 1A to 1C.

With reference to FIG. 1A, the sample collection device 10 includes a collecting member in the form of a swab 11, an indicator pad 12 and an elongated capillary tube 13 providing fluidic communication between the swab 11 and the indicator pad 12.

In this embodiment of the invention the mean cross-section of the indicator pad 12 is less than the mean cross-section of the swab 11. With reference to FIG. 1B, the capillary tube 13 has a bore 131 which is sufficiently narrow to provide a capillary effect. In some embodiments of the invention the bore 131 may contain sponge material taking up part of the volume of the bore 131. In this embodiment of the invention the capillary tube is made of polar fused silica tubing having wall thickness of 0.1 mm, a diameter of 0.25 mm and a length of 6 cm. It will be appreciated that the invention is not limited to such dimensions.

The swab 11 is formed of a spongeous material which can readily absorb sample fluid from a sample source and which may also readily release the sample fluid for analytical purposes through for example, the application of mechanical pressure or capillary transfer. Such material may be, for example cotton wool, hydrophilic or hydrophilized synthetic polymer materials, such as polyethylene vinylacetate, as well as other polyesters, and polypropylene. Material reagents such as for example, buffering compounds to adjust the pH of the test liquid, or compounds for eliminating potentially interfering substances present in the sample fluid may be added to the swab 11 where appropriate.

The indicator pad 12 includes a chemical component which changes colour on contact with the sample fluid to be collected. The sample fluid may be a liquid biological sample such as saliva, blood or urine or other bodily fluids. The sample fluid may or may not be treated prior to being collected.

With reference to FIG. 1C, the components of FIG. 1A are housed in an elongated housing 14. The housing 14 includes a handle 15 for manipulation of the device and a flank 16. The device is also provided with a sealing ring 18 to provide a seal between the collection device and a sample test device during transfer of the sample fluid from the sample collection device to the sample test device. The handle 15, can for example be provided with a colour which can serve for sample identification and thus facilitate handling of larger numbers of different samples.

The indicator element 12 is disposed in the handle 15 and may be viewed from outside the housing via a window 17 provided on the wall of the handle 15. The swab 11 is located at the opposing end of the housing 14 from the indicator pad 12, and is exposed to the exterior of the housing 14 for the retrieval of a sample fluid. A cover may be provided to place over the swab 11 when the device is not in use.

It will be appreciated that in alternative embodiments of the invention, the sample collecting member may be made of non-absorptive material and arranged to collect and release a fluid sample by means of its shape or construction. In some embodiments the device may comprise an indicator chamber comprising the indicator element and a collecting chamber comprising a collecting element with a capillary channel in fluidic communication between the collecting chamber and the indicator chamber.

In use, for instance in the collection of saliva samples, the sample collection device is held in the hand of the user using the handle 15 and the swab 11 is placed in the sample source, for example, in the oral cavity of a person from which the saliva sample is to be retrieved. In alternative applications, the swab 11 may be placed in a container containing blood or urine, for example.

The fluid sample is absorbed from the sample source by the collector swab 11 and an amount of the absorbed fluid is transferred from the swab 11 via the capillary tube 13 to the indicator pad 12. The chemical reagent in the indicator pad 11 changes colour on contact with the sample fluid to indicate when a sufficient volume of the sample fluid has been collected for analytical purposes. The change of colour can be witnessed through the window 17 of the housing 14.

The elongated capillary channel 13 enables the indicator pad 12 to be located at a distance from the collecting swab 11 while requiring only a minimum amount of sample fluid for indication purposes. This can be useful for the user, especially when collecting saliva, since he can read the indicator element 12 while he has the sample collection device in his mouth, and therefore can stop collecting saliva as soon as the indication is given. This helps to speed up the sample collecting step and to minimise the amount of sample to be collected.

Moreover, since the fluid sample is transferred to the indicator pad in a capillary tube, only a very small quantity of the fluid sample is necessary to perform an adequacy indication and dead volume is reduced. Due to the capillary effect, the capillary tube immediately transports the right quantity of sample to the indicator pad. The capillary tube is less cumbersome and may be provided over a relatively long distance thereby facilitating visual accessibility to the indicator element. The diameter of the capillary tube may be adapted according to its length and the hydrophilicity of its inner wall. Furthermore, the inner wall of the capillary element may be treated such that their hydrophilicity is improved, by techniques well known by the skilled person in the art.

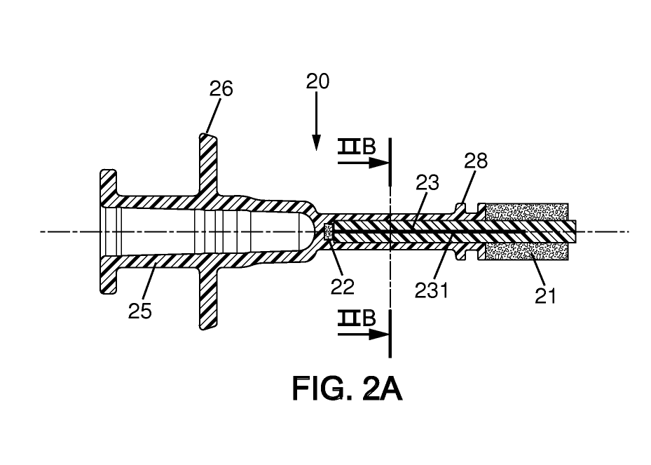

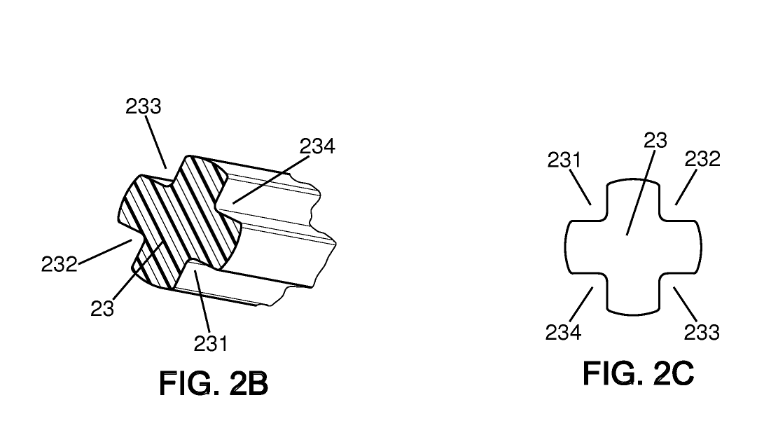

A sample collection device according to a second embodiment of the invention will be described with reference to FIGS. 2A and 2B. The sample collection device 20 includes a collecting member in the form of a swab 21, an indicator pad 22 provided with an indicating dye and an elongated connecting rod 23 provided with capillary channels for providing fluidic communication between the swab 21 and the indicator pad 22. The swab 21 is arranged to surround at least part of the connecting rod 23 providing contact between the swab 22 and the capillary channels.

With reference to FIG. 2B, in this embodiment four capillary channels 231, 232, 233 and 234 are provided on the exterior surface of the connecting rod 22, and extend along the connecting rod in the same axial direction from the swab 21 to the indicator pad 22. The connecting rod 23 may be made from Poly Methyl Methacrylate (PMMA) material and the capillary channels are sufficiently narrow to provide capillary effects. Although in this embodiment four capillary channels are provided on the exterior surface of the rod, it will be appreciated that in alternative embodiments any number of capillary channels extending from the collector swab 21 to the indicator pad 22 may be provided on the connecting rod 23.

The swab 21 is similar to the swab 11 of the previous embodiment and is made of spongeous absorptive material. The indicator pad 22 may be provided with a water soluble dye which on contact with the saliva sample provides a colour indication that a sufficient quantity of saliva sample has been collected.

The connecting rod 23 is disposed by a friction fit in the housing 24. The capillary paths from the swab 21 to the indicator pad 22 are defined by the inner wall of the housing 24 and the capillary channels 231, 232, 233 and 234 of the connecting rod 23. In alternative embodiments of the invention, the connecting rod 23 may be a snap fit or a click fit with the housing 24.

In use, for example in the collection of saliva samples, the device is held in the hand of the user using the handle 25 and the swab 21 is placed in the sample source, for example in the oral cavity of a person from which the saliva sample is to be retrieved. In alternative applications, the swab 21 may be placed in a container containing blood or urine for example.

The fluid sample collected by the collector swab 21 is transferred via the capillary channels 231-234 to the indicator pad 22. The dye in the indicator pad 21 portrays a colour to indicate when a sufficient volume of the fluid sample has been collected for analytical purposes. The colour can be witnessed through a window of the housing 24 positioned at the level of the indicator pad 22. As in the previous embodiment the transport of fluid by capillary effects can lead to a reduction in dead volume and collection time, and enables the indicator element to be located distal to the sample collecting member and more easily accessed visually.

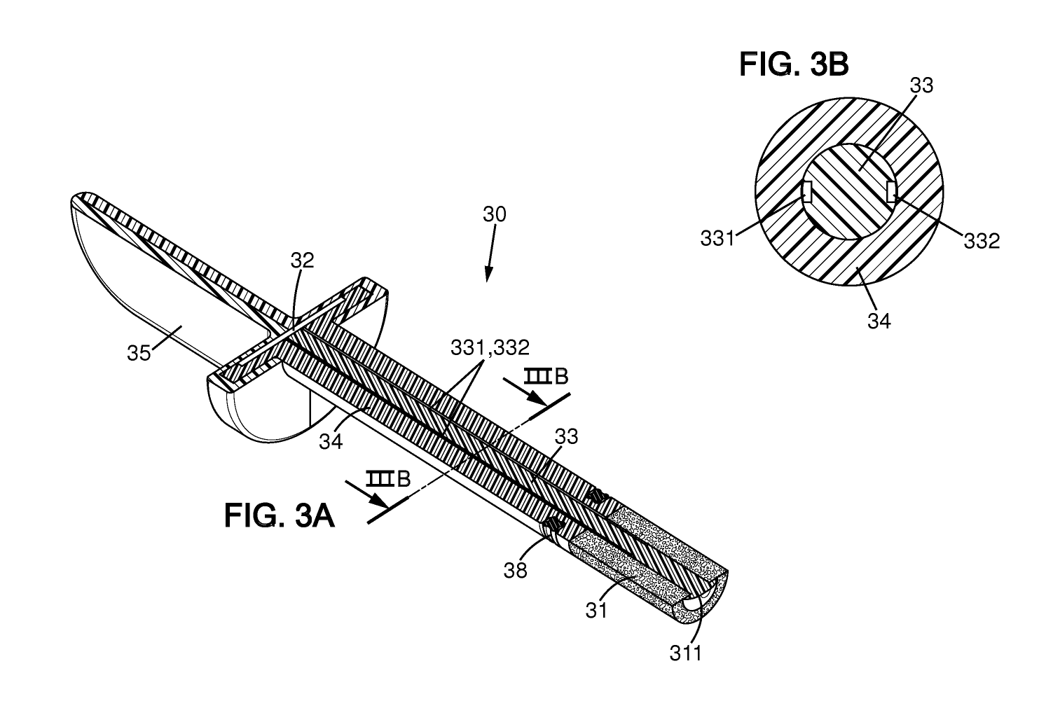

A sample collection device according to a third embodiment of the invention will be described with reference to FIGS. 3A and 3B.

With reference to FIG. 3A, the sample collection device 30 includes a collecting member in the form of a swab 31, an indicator swab 32 and an elongated connecting rod 33 having a pair of capillary channels 331 and 331 defined on the external surface of the rod 33 extending along the rod 33 in an axial direction. The sample device includes a housing 34 in which the connecting rod 33 and the indicator swab 32 are disposed. Elongated capillary paths are thus defined between inner wall of the housing 34 and the capillary channels 331 and 332 providing fluidic communication between the collecting swab 31 and the indicator swab 32. A cover plate 311 is provided at the end of the capillary paths in contact with the swab 31 to prevent leakage from the capillary paths to the exterior of the housing 34.

In use, for example in the collection of saliva samples, the device is held in the hand of the user using the handle 35 and the swab 31 is placed in the sample source, for example in the oral cavity of a person from which the saliva sample is to be retrieved. In alternative applications, the swab 31 may be placed in a container containing blood or urine for example.

The fluid sample collected by the collector swab 31 is transferred via the capillary channels 331 and 332 to the indicator pad 32. The dye in the indicator pad 32 portrays a colour to indicate when a sufficient volume of the sample has been collected for analytical purposes. The colour can be witnessed through a window (not shown) of the housing 34 positioned at the level of the indicator pad 32. As in the previous embodiments the transport of fluid by capillary effects can lead to a reduction in dead volume and collection time, and enables the indicator element to be located distal to the sample collecting member and more easily accessed visually.

In another embodiment of the present invention the sample collector is provided with a connecting rod between the handle and the collecting member, which facilitates handling of the sample collector.

In order for the collected fluid sample to be analysed, the collected sample is transferred from the collection device to a sample test device. For this purpose the sample collection device according to any of the foregoing embodiments may be arranged to co-operate with a counterpart receiving member of a said test device. The sample collection device may be further provided with retaining members such as clipping means to prevent it being pulled away from the receiving member of the test device during transfer of the collected fluid sample from the sample collection device to the sample test device.

The receiving member (not shown) of such an analysing system with which the collection device may be employed may comprise:
a hollow portion guiding the sample collection device and
a sample collecting chamber having an inlet designed for squeezing the collecting member of the sample collection device when the sample collection device is pushed in the hollow portion beyond a first position.

Downstream of the collecting chamber, the receiving member comprises a test element such as a test strip to indicate the presence or absence of an analyte in the sample fluid. The test strip surface is provided with:
immobilised reagent able to specifically bind the analyte, and/or with
analyte derivatives so as to specifically bind with t a mobile reagent attached to the analyte.

The immobilised and/or mobile reagent and/or analyte and/or analyte derivatives may be bound to labels arranged for being detected by detecting means once they are directly or indirectly attached to the test strip surface.

Between the collecting chamber and the test strip, the receiving member may also be provided with driving fluidic elements and/or filters. Preferably, this intermediary zone stores labels and/or mobile specific reagents to be incubated and/or mixed in the sample so as to specifically bind the analyte.

A sample test assembly comprising the receiving member and a sample collection device according to any one of the foregoing embodiments, can be introduced in a data reader which comprises means for detecting the analytes or labels bound to the test strip surface and assessing therefrom an amount of analytes in the sample.

Embodiments of the invention may find application in diagnostic procedures, screening procedures, detection of drug abuse as a drugs abuse sensor, and the like.

Although the present invention has been described hereinabove with reference to specific embodiments, the present invention is not limited to the specific embodiments, and modifications will be apparent to a skilled person in the art which lie within the scope of the present invention as determined by the appended claims. In particular the different features from different embodiments may be interchanged, where appropriate.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A sample collection device (10; 20; 30) for collecting a fluidic biological sample, the device comprising:
a collecting member (11; 21; 31) for collecting the fluidic sample;
an indicator element (12; 22; 32) for providing an indication that a desired amount of the fluidic sample has been collected; and
an elongated capillary element (13; 231, 232, 233, 234; 331,332) for providing fluidic communication between the collecting member (11; 21; 31) and the indicator element (12; 22; 32) such that at least a portion of the fluidic sample may be transferred from the collecting member (11; 21; 31) to the indicator element (12; 22; 32).

2. A sample collection device according to claim 1, wherein the mean cross section of the elongated capillary element (13; 231, 232, 233, 234; 331,332) is significantly less than the mean cross-section of the collecting member (11; 21; 31).

3. A sample collection device according to claim 1, wherein the mean cross section of the collecting member (11; 21; 31) is more than ten times greater than the mean cross-section of the capillary element (13; 231, 232, 233, 234; 331,332).

4. A sample collection device according to claim 1, wherein the capillary element extends along a single line.

5. A sample collection device according to claim 1, wherein the capillary element comprises a tube (13) having an inner bore (131) arranged to provide a capillary effect.

6. A sample collection device according to claim 5, wherein a porous material is provided in at least part of the inner bore (131).

7. A sample collection device according to claim 1, wherein the capillary element (231, 232, 233, 234; 331, 332) comprises a connecting element (23; 33) arranged between the collecting member (21; 31) and the indicator element (22; 32), wherein the outer surface of the connecting element is provided with at least one capillary channel (231, 232, 233, 234; 331, 332) in fluidic communication between the collecting member (11; 21; 31) and the indicator element (12; 22; 32).

8. A sample collection device according to claim 7, wherein the capillary element further comprises means for laterally closing at least one capillary channel (231, 232, 233, 234; 331, 332).

9. A sample collection device according to claim 8, wherein the closing means is an outer shell surrounding the periphery of the connecting element.

10. A sample collection device according to claim 7, wherein the connecting element (23; 33) is a rigid elongated rod.

11. A sample collection device according to claim 1 further comprising a housing lodging at least the indicator element (12; 22; 32), the housing (14; 24; 34) being provided with a window (17) for viewing the indicator element (12; 22; 32).

12. A sample collection device according to claim 1, wherein the volume of the indicator element (12; 22; 32) is significantly less than the volume of the collecting member (11; 21; 31).

13. A sample collection device according to claim 1, wherein the collecting member (11; 21; 31) is operable to release the fluidic sample by application of mechanical pressure.

14. A sample collection device according to claim 1, wherein the sample collection device is operable to cooperate with a receiving member of a sample test device to transfer the fluidic sample into the sample test device and further comprises retaining members for retaining the sample collection device in place relative to the sample test device.

15. A sample collection device according to claim 1, wherein the length of the capillary element (13; 231, 232, 233, 234; 331,332) is chosen such that, in use, when a user collects the fluidic sample via the collecting member (11; 21; 31) he can see the indicator element (12; 22; 32).

16. A sample test assembly comprising the sample collection device (10; 20; 30) according to claim 1 and a receiving counterpart, wherein the receiving counterpart comprises:
a receiving portion arranged to receive the sample collection device (10; 20; 30),
a sample collecting chamber having an inlet arranged to co-operate with the sample collection device (10; 20; 30) so as to release the fluidic sample from the collecting member (11; 21; 31) when the device is received in the receiving portion beyond a relative position,
a test strip in fluidic communication with the sample collecting chamber, provided with biochemistry arranged for allowing detectability of at least one analyte in the fluidic sample.
